(19) United States
(12) Patent Application Publication
(10) Pub. No.: US 2010/0137721 A1
(43) Pub. Date: Jun. 3, 2010

(54) TRANSURETHRAL ULTRASONIC IMAGING SYSTEM

(76) Inventors: Harold B. Rose, Tarzana, CA (US); Robert M. Woskow, Encino, CA (US); Warren G. Grundfest, Los Angeles, CA (US); Spencer L. MacKay, Agoura Hills, CA (US); David J. Rubenstein, Woodland Hills, CA (US); James J. Elist, Beverly Hills, CA (US); Alan D. Crawford, Burbank, CA (US)

Correspondence Address: CHRISTIE, PARKER & HALE, LLP, PO BOX 7068, PASADENA, CA, 91109-7068, US

(21) Appl. No.: 12/703,696

(22) Filed: Feb. 10, 2010

Related U.S. Application Data
(62) Division of application No. 11/118,166, filed on Apr. 29, 2005, now abandoned.

(51) Int. Cl. A61B 8/14

(52) U.S. Cl. 600/463

(57) ABSTRACT

An ultrasound scanning system and methods of using the same. In one preferred form, an ultrasound scanning system comprises an acoustic imaging catheter comprising an ultrasonic transducer, a motion control system and an imaging computer system for imaging a patient's genitourinary system. In another preferred form, an ultrasound scanning system is used for imaging a patient's prostate gland.

### CROSS-REFERENCE TO RELATED APPLICATION(S)

This application is a divisional application of, and claims priority to and the benefit of, U.S. patent application Ser. No. 11/118,166, entitled “Transurethral Ultrasonic Imaging System,” filed Apr. 29, 2005, the entire content of which is incorporated herein by reference.

### FIELD OF THE INVENTION

The field of the present invention is imaging systems and their methods of use, and more particularly, ultrasound scanning systems and their use in imaging a patient's genitourinary system.

### BACKGROUND OF THE INVENTION

Ultrasound is an imaging technique, which uses high-frequency sound waves to produce images of the organs and structures of the body. The ultrasound technique involves sending sound waves into the body. These sound waves reflect off the internal organs and are recorded by special instruments that create images of the anatomic parts of the internal organs. The ultrasound technique uses no ionizing radiation, and provides real-time imaging. Ultrasound is used to detect and monitor the growth of the fetus, examine many of the body's internal organs, for example, heart, liver, gallbladder, spleen, pancreas, kidneys, and bladder.

Recently, ultrasound has been used to detect possible disorders within a man's prostate gland. For example, those skilled in the art will note that transabdominal ultrasound (TAUS), transperineal ultrasound (TPUS), transrectal ultrasound (TRUS) and transurethral ultrasound (TUUS) scanning systems have been used to examine prostate gland abnormalities, such as benign prostatic hyperplasia (BPH), carcinoma of the prostate gland (CAP), prostatitis, prostatic abscess, and prostatic calculi.

TRAS and TPUS are non-invasive and do not require any special patient preparation. However, TRAS and TPUS fail to produce high-quality images of the prostate gland.

Since the introduction of the prostate-specific antigen (PSA) screening test and early detection of prostate cancer, the role of TRUS has changed. TRUS is mainly used to image the prostate gland and aid in guided needle biopsy. TRUS scanning system consists of an ultrasonic transducer and a monitoring system. The ultrasonic transducer is a small, cylinder-shaped probe, which is lubricated and inserted into the rectum to view the prostate gland. The ultrasonic transducer functions as both a loudspeaker (to transmit the sounds) and a microphone (to record the sounds). When the ultrasonic transducer is inserted into the rectum it directs a stream of inaudible, high frequency sound waves into the body. As the sound waves echo back from the body's fluids and tissues, the sensitive microphone in the ultrasonic transducer records the strength and character of the reflected waves. The ultrasound image is immediately visible on a nearby screen that looks much like a computer or television monitor. If a suspicious lesion is identified with TRUS, a biopsy can be performed. However, TRUS cannot be completely relied upon for accurate imaging of the entire prostate gland.

TUUS is generally used as a guide to remove overgrown prostate tissue with a laser beam. However, it will be noted by those skilled in the art that most current medical interventional procedures, for example, surgery, biopsy, and ablation still require “blind” approaches, i.e., the clinicians cannot directly see the target and/or pathway to the target during the ultrasound imaging of the prostate gland. A visualization of the target during ultrasound scanning of the prostate gland, if available, are often limited to two-dimensional, slow and/or off-line displays.

Because it is desirable in many applications, to have improved ultrasonic images of the prostate gland, both in individual transverse sections and in rendered three-dimensional presentations of the entire prostate gland, while at the same time facilitating digitally-positioned targeted biopsies based on image-apparent focal tissue abnormalities, a new ultrasound scanning system is desired.

### SUMMARY OF THE INVENTION

The present invention is directed to an ultrasound scanning system for the transurethral imaging of a patient's genitourinary system, and in one embodiment, the male prostate gland. The ultrasound scanning system is capable of three-dimensional imaging of the patient's prostate gland, is capable of producing multiple arrays of transverse slice images of the selected section of the patient's prostate gland, which results in complete scanning of the patient's prostate gland, and is capable of facilitating digitally positioned targeted biopsies based on the image-apparent focal tissue abnormalities, potentially reducing the required number of tissue biopsy samples. In one preferred form, the ultrasound scanning system for imaging the patient's prostate gland comprises an acoustic imaging catheter, a motion control system, and a computer system. The acoustic imaging catheter is capable of being inserted within the patient's prostatic urethra. The acoustic imaging catheter comprises an ultrasonic transducer. The ultrasonic transducer is rotated inside the acoustic imaging catheter, which enables production of the scan data representative of a section of the patient's prostate gland. The acoustic imaging catheter is moved to different positions inside the patient's prostatic urethra, in relation to a fixed anatomical landmark, to generate images of the selected sections of the patient's prostate gland. The motion control system controls the axial and rotational motion of the acoustic imaging catheter. The computer system is in communication with the acoustic imaging catheter and the motion control system. The computer system processes signals received from the acoustic imaging catheter and generates selected images including, if desired, a three-dimensional image of the patient's prostate gland, which is displayed on an associated image viewing device. The image viewing device is positioned in a location, which allows substantially simultaneous viewing of both a patient's pelvic region and the image viewing device to the physician, while ultrasonically imaging the patient's prostate gland.

It follows that the ultrasound scanning system embodying a preferred form of the present invention is capable of three-dimensional imaging of the patient's prostate gland. Further, because the acoustic imaging catheter is moved to different positions inside the patient's prostatic urethra in relation to a fixed anatomical landmark, the ultrasound scanning system embodying a preferred form of the present invention is capable of producing multiple arrays of transverse slice images of the selected sections of the patient's prostate gland. This provides a complete scanning of the patient's prostate gland. Further, because the image data of the patient's prostate gland is stored in the computer system, the ultrasound scanning system, in accordance with the present invention, provides the retrieval of the required information anytime. Further, the ultrasound scanning system embodying a preferred form of the present invention facilitates digitally positioned targeted biopsies based on the image-apparent focal tissue abnormalities, potentially reducing the required number of tissue biopsy samples.

Accordingly, it is an object of the present invention to provide an improved ultrasound scanning system which is capable of producing a three-dimensional rendering of the patient's genitourinary system.

It is a further object of the present invention to provide an improved ultrasound scanning system which is capable of producing a three-dimensional rendering of the patient's prostate gland.

It is still another object of the present invention to provide for a complete scanning of the patient's prostate gland an improved ultrasound scanning system which is capable of producing multiple arrays of transverse slice images of the selected section of the patient's prostate gland.

It is yet another object of the present invention to provide for facilitating digitally positioned targeted biopsies based on the image-apparent focal tissue abnormalities an improved ultrasound scanning system which is capable of imaging the patient's prostate gland.

Exemplary embodiments of the present invention that are shown in the drawings are summarized below. These and other embodiments are more fully described in the Detailed Description section. It is to be understood, however, that there is no intention to limit the invention to the forms described in this Summary of the Invention or in the Detailed Description. One skilled in the art can recognize that there are numerous modifications, equivalents and alternative constructions that fall within the spirit and scope of the invention as expressed in the claims.

### BRIEF DESCRIPTION OF THE DRAWINGS

FIG. 1 illustrates an ultrasound scanning system for imaging a patient's genitourinary system, in accordance with an embodiment of the invention.

FIG. 2 illustrates a remote keypad for controlling the imaging of a patient's genitourinary system, in accordance with an embodiment of the invention.

FIG. 3 illustrates a motion control system for controlling the axial and rotational motion of an acoustic imaging catheter, in accordance with an embodiment of the invention.

FIG. 4 illustrates a sectional anatomical view showing an acoustic imaging catheter within a patient's prostatic urethra, in accordance with an embodiment of the invention.

FIG. 5 is a flowchart illustrating a method for imaging a patient's prostate gland, in accordance with an embodiment of the invention.

### DETAILED DESCRIPTION

For the sake of convenience, the terms used to describe various human anatomical structures and embodiments of the invention are defined below. It should be understood that these are provided merely to aid the understanding of the description, and that the definitions should in no way limit the scope of the invention, which is defined by the appended claims.

Anterior: Situated at the front or the front surface of an organ.

Apex of the prostate: The end of the prostate gland located farthest away from the urinary bladder.

Axial/Longitudinal: Along the centerline of the urethra, regardless of patient position.

Biopsy: The removal of small sample(s) of tissue for examination under a microscope.

Bladder: The hollow organ that stores and discharges urine from the body.

Bladder neck: The outlet area of the bladder. It is composed of circular muscle fibers (bladder sphincter), and helps control urine flow from the bladder into the urethra.

Catheter drive mechanism: A motion control system that can provide axial and/or rotational motion to an imaging catheter, or an ultrasonic transducer disposed within an imaging catheter.

Distal: Remote, farther from any point of reference (the opposite of proximal).

Genitourinary system: Pertaining to the genital and urinary systems.

Imaging catheter: A tubular mechanism, containing an ultrasonic transducer for organ-tissue imaging.

Inferior: Anatomically refers to a lower surface of an organ, or a location situated below a given reference point.

Introducer: A device that facilitates the insertion of a catheter into the urethra.

Posterior: Situated at the back or the back surface of an organ.

Prostatic Urethra: The segment of the urethra, which is surrounded by prostatic tissue from the proximal end at the bladder neck to the distal end at the apex of the prostate gland.

Proximal: Closer to any point of reference.

Superior: Anatomically refers to an upper surface of an organ, or situated above a given reference point.

Transducer: A device, which transforms one form of energy to another form of energy (e.g. electrical to acoustical energy, or, conversely, acoustical to electrical energy).

Transurethral: A procedure performed through the urethra.

Transverse: Placed crosswise, situated at right angles to the long axis of an organ.

Various embodiments of the invention comprise an ultrasound scanning system and a method for the transurethral imaging of a patient's genitourinary system, and in one embodiment, the male prostate gland. The ultrasound scanning system includes an acoustic imaging catheter, a motion control system and an imaging computer system. The acoustic imaging catheter is moved inside a patient's urethra. The motion control system controls the axial and rotational motion of the acoustic imaging catheter inside the patient's urethra.

In one presently preferred embodiment, an ultrasonic transducer is rotated inside the acoustic imaging catheter for scanning the patient's prostate gland. The acoustic imaging catheter is moved to different positions inside the patient's urethra in relation to the neck of the patient's urinary bladder. Scanning in this fashion produces transverse slice images of the selected sections of the patient's prostate gland. The imaging computer system processes the transverse slice image signals received from the acoustic imaging catheter, and generates selected images including, if desired, a three-dimensional image of the patient's prostate gland. The three-dimensional image is displayed on an image viewing device, such as a CRT, LCD, or other display.

The system elements, method steps and various embodiments of the invention are described in detail with reference to the appended drawings and flowcharts.

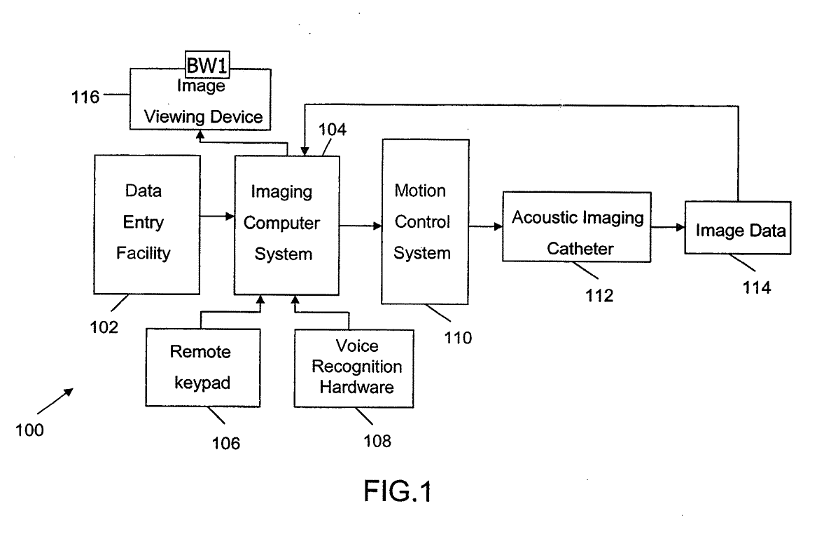

FIG. 1 illustrates an ultrasound scanning system 100 for imaging a patient's genitourinary system, in accordance with an embodiment of the invention. The ultrasound scanning system 100 may comprise a data entry facility 102, for example, a keyboard, keypad, magnetic card reader, optical scanner, or other data entry device coupled to a personal computer, server, or other data processing system; an imaging computer system 104, for example, a central processing unit, personal computer, server, or other data processing system; a remote keypad 106; voice recognition hardware 108 and related software for interfacing with the imaging computer system 104; a motion control system 110 that communicates with the imaging computer system 104; an acoustic imaging catheter 112 configured to interface with the motion control system 110; and an image viewing device 116, such as a CRT, LCD, or other monitor or display device that can display image data 114, rendered by the imaging computer system 104. In one preferred embodiment, the data entry facility 102 and the imaging computer system 104 may be connected via a data communications network, such as a local area network (LAN), which is not shown.

The patient data is entered in the data entry facility 102. In accordance with one embodiment of the invention, the data entry facility 102 is powered by a Pentium 2 processor or better. However, the invention should not be construed to be limited to the use of a Pentium 2 processor only. In various embodiments of the invention, the data entry facility 102 may include any processor-containing device, such as a mainframe computer, personal computer, laptop, notebook, microcomputer, server, personal data manager or ‘PIM’ (also referred to as a personal information manager), or any of the like, without deviating from the scope of the invention. The data entry facility 102 may comprise any of a number of operating systems that are currently available on the market including, but not limited to, Microsoft Windows 98, XP, or ME, NT, 2000, Linux, Unix, or any of the like. The data entry facility 102 also may comprise a non-volatile data storage facility, such as a read-and-write-capable CD ROM drive or DVD drive or, alternatively, some type of magnetic media facility.

The patient data entered into the data entry facility 102 will typically comprise patient-related information, such as the name of the patient, age of the patient, patient identification number, administrative data, referring physician's orders, patient diagnosis, disease symptoms, and the like.

The data entry facility 102 may forward the patient data to the imaging computer system 104 via some type of communications network, such as a local area network (LAN) (not shown). Alternatively, the patient data may be transferred from the data entry facility 102 to the imaging computer system 104, through the use of non-volatile storage media, such as magnetic or optical storage media that may be included within a patient file.

The imaging computer system 104 processes the patient data received from the data entry facility 102. In one preferred embodiment of the invention, the imaging computer system 104 is configured to receive the patient data directly from the data entry facility 102, or indirectly via the above-described storage media; it is configured to capture and store sonographic image data representative of the patient's prostate gland; it is configured to render two-dimensional and three-dimensional images of the patient's prostate gland on an image viewing device 116; it is configured to store relevant image data on non-volatile media that can be included in the patient's file; and it is configured to control the motion control system 110.

As explained above, in accordance with one embodiment of the invention, the data entry facility 102 may include various data storage facilities for storing the patient image data on optical, magnetic, or other media. The storage media may include, for example, various hard disk variants, floppy/compact disk variants, digital versatile disk (DVD) variants, smart cards, partially or fully-hardened removable media, read-only memory, other non-volatile media, random-access memory, cache memory, and the like.

In accordance with one embodiment of the invention, the imaging computer system 104 includes a program for controlling the motion control system 110, which includes any suitable locally or remotely executable program or sequence of coded instructions, which enables the imaging computer system 104 to control the motion control system 110. The imaging computer system 104 further includes the programming for capturing and storing sonographic image data representative of the patient's prostate gland, and for rendering two-dimensional and three-dimensional images of the patient's prostate gland on an image viewing device 116.

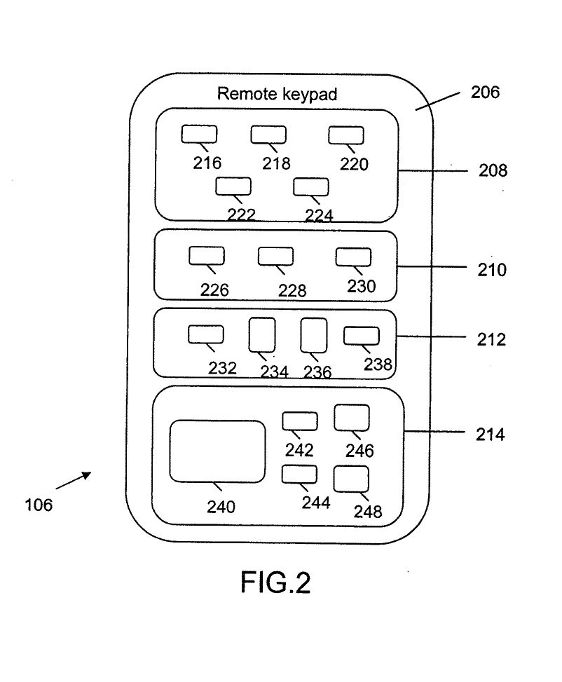

In accordance with another embodiment of the invention, the imaging computer system 104 includes the remote keypad 106 and the voice recognition hardware 108. The remote keypad 106 is described in detail in conjunction with FIG. 2. The voice recognition hardware 108 includes the hardware and software for processing speech signals for controlling the motion control system 110, and for controlling the imaging process.

In several embodiments of the invention, the imaging computer system 104 is connected to the motion control system 110. However, those skilled in the art will appreciate that the imaging computer system 104 and the motion control system 110 may, if desired, comprise a single unit. The motion control system 110 controls the axial and rotational motion of the acoustic imaging catheter 112, and/or the axial and rotational motion of an ultrasonic transducer that forms a part of the acoustic imaging catheter 112.

The acoustic imaging catheter 112 preferably includes the ultrasonic transducer (not shown), which scans the prostate gland or other area of interest of the patient. The acoustic imaging catheter 112 is moved to different positions inside a patient's urethra. As the acoustic imaging catheter 112 is moved to different positions inside the patient's urethra, it performs a radial scan of any surrounding tissue and transmits received signals to the imaging computer system 104, which in turn, captures and stores data representative of the cross-sectional images of the surrounding tissue.

In one preferred form, the captured image data 114 can be used to render multiple (typically, 160 or more) transverse (cross-sectional) specific slices of the prostate gland of the patient. The slices when processed and combined can be used to render 360° imaging of the entire prostate gland of the patient. Thus, the imaging computer system 104 may process signals received from the acoustic imaging catheter 112 and generate two-dimensional and three-dimensional images of the patient's prostate gland for display on the associated imaging device 116. The image viewing device 116 preferably is positioned in a location, which allows substantially simultaneous viewing of both the patient's pelvic region and the image viewing device 116 for the convenience of the physician while ultrasonically imaging the patient's prostate gland.

FIG. 2 illustrates a remote keypad 106 that may be used to control the imaging of a patient's genitourinary system, in accordance with various embodiments of the present invention. The remote keypad 106 preferably includes a spill-proof membrane keypad 206 and a display screen 240. The remote keypad 106 is preferably coupled to the imaging computer system 104 through a standard wired or wireless interface. For example, in a wireless environment, the remote keypad 106 may communicate with the imaging computer system 104 over a Bluetooth or 802.11 interface, and in a wired embodiment the remote keypad 106 may communicate with the imaging computer system 104 over, for example, a conventional Ethernet connection.

In accordance with one embodiment of the invention, the spill-proof membrane keypad 206 includes a scan control menu bar 208, an image control menu bar 210, a motion control menu bar 212, and a user option control menu bar 214. Thus, the remote keypad 106 includes functional groupings. The scan control menu bar 208 includes a key 216, a key 218, a key 220, a key 222 and a key 224 for controlling the scanning of the selected section of the patient's prostate gland. In accordance with one embodiment of the invention, the key 216 is used to give a command for starting the scanning of the patient's prostate gland. The key 218 is used to give a command for pausing/resuming the scanning of the selected section of the patient's prostate gland. The key 220 is used to give a command for stopping the scanning of the selected section of the patient's prostate gland. The key 222 is used to give a command for controlling the scanning of the selected section of the patient's prostate gland, as the acoustic imaging catheter 112 is retracted away from a fixed anatomical landmark. The key 224 is used to give a command for controlling the scanning of the selected section of the patient's prostate gland, as the acoustic imaging catheter 112 is advanced towards the fixed anatomical landmark. When imaging the patient's prostate gland, the fixed anatomical landmark preferably is the neck of the patient's urinary bladder. In accordance with one embodiment of the invention, the remote keypad 106 includes a flash-reprogrammable microcontroller.

The image control menu bar 210 includes a key 226, a key 228, and a key 230 for performing a plurality of functions, such as inverse video, marking of images, and switching to manual scanning of the patient's prostate gland. The key 226 is used to render a reverse image of an area of interest. In such a situation, the black areas of an image will be white, and the white areas of an image will be black. Similarly, the light grey areas and dark grey areas will be inverted. The key 228 is used to mark or flag selected images for later viewing and analysis, and the key 230 is used to create filenames for various imaging sessions, imaging files, and specific rendered images, if desired. Further, by marking a filename, extra data, such as metadata, may be associated with a particular imaging file.

The motion control menu bar 212 includes a key 232, a key 234, a key 236, and a key 238 for controlling the motion of the acoustic imaging catheter 112 within the patient's urethra. In accordance with one preferred embodiment of the invention, the key 232 is used to give a command for retracting the acoustic imaging catheter 112 within the patient's urethra in such a manner that it is pulled away from the neck of the patient's urinary bladder towards an apex of the patient's prostate gland. Thus, the acoustic imaging catheter 112 is retracted across the full length of the patient's prostate gland. The key 234 is used to give a command for retracting the acoustic imaging catheter 112 in a stepwise manner for a predetermined distance. It will be apparent to persons skilled in the art that the acoustic imaging catheter 112 is retracted in steps inside the patient's urethra, in relation to the neck of the patient's urinary bladder. The key 236 is used to give a command for advancing the acoustic imaging catheter 112 from the apex of the patient's prostate gland towards the neck of the patient's urinary bladder in a stepwise manner for a predetermined distance. The key 238 is used to give a command for advancing the acoustic imaging catheter 112 within the patient's postatic urethra across the full length of the patient's prostate gland.

The user option control menu bar 214 includes a display screen 240, a key 242, a key 244, a key 246, and a key 248. In accordance with one embodiment of the invention, the display screen 240 is a CRT, LCD, vacuum fluorescent display screen or other display type, which displays functions, such as set up controls, parameter adjustments, run-time status and user help. For example, the setup controls may display the controls for adjusting parameters, such as sharpness of the image, focus of the image, brightness of the image, and the like. The run-time status may display the progress of the given commands, for example, the time of scanning. The key 242 is used to give a command for selecting the desired function. The key 244 is used to give a command for scrolling through a menu of available functions to the desired function. The key 246 is used to give a command to notify the imaging computer system 104 that a scan of the patient's prostate gland is complete, and that no additional imaging will be performed at this time so that the data imaging process is finalized; any further activity will require explicit operator action so as not to disturb the already-acquired data. The key 248 is used to give a command for the emergency stopping of all procedural actions of the ultrasound scanning system 100 on the patient's prostate gland, in case of some either system or patient situation.

In accordance with one embodiment of the invention, the remote keypad 106 has been described with the help of a limited number of keys for controlling the imaging of the patient's prostate gland. It will be apparent to a person skilled in the art that a multiplicity of keys may be used for a plurality of functions. Additionally, the operations of the remote keypad 106 may be performed utilizing a mouse and keyboard, or any other interactive method directly or indirectly attached to the imaging computer system 104 itself.

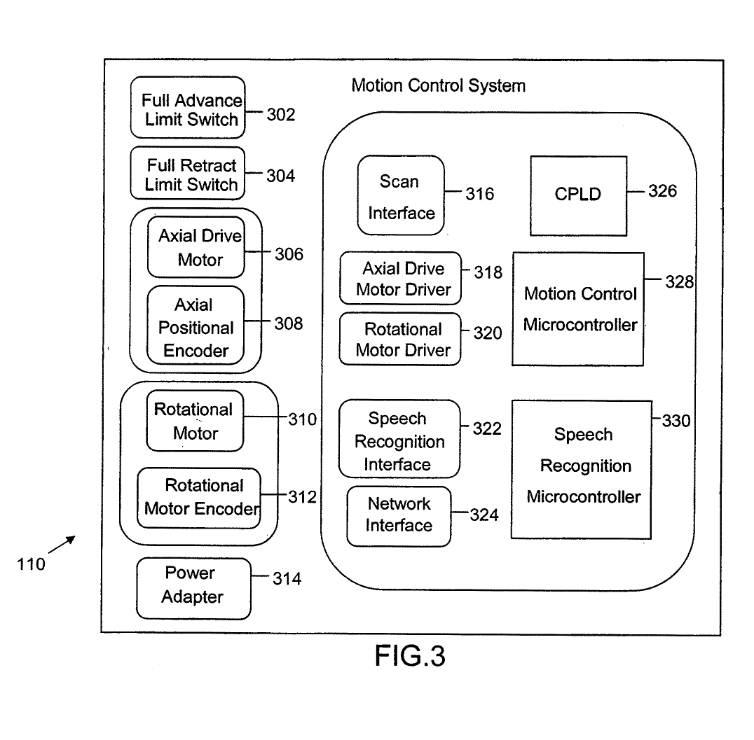

FIG. 3 illustrates the motion control system 110, in accordance with an embodiment of the invention. The motion control system 110 includes a full advance limit switch 302, a full retract limit switch 304, an axial drive motor 306, an axial positional encoder 308, a rotational motor 310, a rotational motor encoder 312, a power adapter 314, a scan interface 316, an axial drive motor driver 318, a rotational motor driver 320, a speech recognition interface 322, a network interface 324, a CPLD 326, a motion control microcontroller 328, and a speech recognition microcontroller 330.

In accordance with one embodiment of the invention, the motion control system 110 controls axial and rotational motion of the acoustic imaging catheter 112, and/or axial and rotational movement of the ultrasonic transducer disposed in the acoustic imaging catheter 112. The axial drive motor 306 provides an axial drive motion to the acoustic imaging catheter 112 inside the patient's urethra. In an embodiment of the invention, the axial drive motor 306 is a stepper motor. A stepper motor is an electromagnetic device that converts digital pulses into mechanical rotations. However, the invention should not be construed to be limited to the use of stepper motors only. Other types of motors, such as brushed direct current motors, brushless direct current motors, alternating current induction motors, servo motors, brushless servo motors, or any of the like can also be used, without deviating from the scope of the invention.

The axial positional encoder 308 is used to monitor the motion of the axial drive motor 306. The imaging computer system 104 uses data generated by the axial positional encoder 308 to maintain the positional integrity of the system and, in particular, the ultrasonic transducer inside the acoustic imaging catheter 112 within a patient's urethra.

The rotational motor 310 provides a rotational motion to the acoustic imaging catheter 112 inside the patient's prostatic urethra, and/or a rotational movement of the ultrasonic imaging transducer disposed in the acoustic imaging catheter 112. In an embodiment of the invention, the rotational motor 310 is a brushed direct current motor. However, the invention should not be construed to be limited to the use of brushed direct current motors only. Other types of motors, such as brushless direct current motors, alternating current induction motors, servo motors, brushless servo motors, or any of the like can also be used without deviating from the scope of the invention.

The acoustic imaging catheter 112 or, more specifically, the ultrasonic transducer (not shown) disposed within the acoustic imaging catheter 112, is rotated at a selected uniform speed inside the patient's urethra. In an embodiment of the invention, the acoustic imaging catheter 112 is rotated at a speed of 100 rpm. However, the persons skilled in the art would realize that the rotational speed of the acoustic imaging catheter 112 is not limited to 100 rpm only. The acoustic imaging catheter 112 can be rotated at any selected rotational speed, without deviating from the scope of the invention. Rotating the ultrasonic transducer inside the acoustic imaging catheter 112 enables the production of the image data 114 representative of a selected segment of the patient's prostate gland.

The rotational motor encoder 312 outputs positional information about the rotational angle and speed of the rotational motor 310. The imaging computer system 104 uses signals generated by the rotational motor encoder 312 to orient a superior aspect of the image of the patient's prostate gland at the top of a display screen on the image viewing device 116. The scan interface 316 facilitates interaction of the motion control system 110 and the imaging computer system 104. Thus, the imaging computer system 104 may be configured to interact with the motion control system 110 to ensure not only proper placement of the ultrasonic transducer (not shown) within a patient's urethra, but also proper scanning of the selected sections of the patient's prostate gland.

The axial drive motor driver 318 drives the axial drive motor 304. The axial drive motor driver 318 utilizes command signals received from the motion control microcontroller 328 to supply the power necessary to energize the axial drive motor 306 windings in the required manner. In accordance with one embodiment of the invention, the axial drive motor driver 318 is a stepper motor driver.

The rotational motor driver 320 drives the rotational motor 310. The rotational motor driver 320 utilizes the command signals received from the motion control microcontroller 328 to supply the power necessary to energize the rotational motor 310 windings in the required manner. It will be apparent to a person skilled in the art that the invention is capable of using numerous types of drivers, for example, unipolar motor drivers, R/L motor drivers, and bipolar motor drivers with different current/amperage ratings and construction technology, without deviating from the scope of the invention.

The speech recognition interface 322 enables the interaction of the motion control system 110 with the voice recognition hardware 108. In accordance with one embodiment of the invention, the speech recognition interface 322 includes a master microcontroller for enabling efficient communication between the motion control system 110 and the voice recognition hardware 108. The speech recognition interface 322 includes a speaker-independent speech processor (although a speaker-dependent speech processor may be used in another embodiment) for processing the speech signals controlling the entire scan process. In various embodiments of the invention, the speech recognition interface 322, the motion control system 110, the motion control microcontroller 328 or the imaging computer system 104 may provide audible confirmation once the speech signals are processed. In various embodiments of the invention, the speech recognition interface 322 also provides for the control of the entire imaging process.

The network interface 324 enables the interaction between the motion control system 110 and the imaging computer system 104. In accordance with one embodiment of the invention, the network interface 324 is 10/100 Mb LAN interface.

In accordance with one embodiment of the invention, the complex programmable logic device (CPLD) 326 is a type of integrated circuit that preprocesses data related to the controlling motion of the axial drive motor 306 and the rotational motor 310. The data related to the controlling motion of the axial drive motor 306 and the rotational motor 310, after being processed by the CPLD 326, is passed on to the motion control microcontroller 328. In an embodiment of the invention, the CPLD 326 outputs the index and quadrature signals to the scan interface 316. In another embodiment of the system, the inputs to the CPLD 326 bypass the CPLD 326, and are fed directly to the motion control microcontroller 328.

The motion control microcontroller 328 provides the step and direction outputs to the axial drive motor driver 318 and speed and directional outputs to the rotational motor driver 320.

In accordance with one embodiment of the invention, a proportional integral derivative control (PID) algorithm maintains the speed of the rotational motor 310. The PID algorithm utilizes index and quadrature data from the rotational motor encoder 312, and provides angular resolution up to 360°/1000, which means that the quadrature encoder signals can resolve up to 1000 angular incremental rotational steps per revolution. In various embodiments of the invention, the rotational motor encoder 312 may include magnetic, optical, Hall-effect or other sensors for resolving the positional information.

In accordance with an embodiment of the invention, the motion control microcontroller 328 has a program module that processes the information related to the action of the axial drive motor 304. This program module provides a linear step resolution of up to 0.000125 inches to the acoustic imaging catheter 112 inside the patient's prostatic urethra.

The motion control microcontroller 328 can also control other functions, such as acceleration, deceleration, steps per second, and the distance traveled of the axial drive motor 306 and the rotational motor 310. The motion control microcontroller 328 includes auxiliary inputs/outputs (I/O) for monitoring the inputs from external sources, such as the full advance limit switch 302 and the full retract switch 304.

In accordance with one embodiment of the invention, the advance limit switch 302 provides a notification to the motion control microcontroller 328 that the axial drive motor 306 has fully advanced the acoustic imaging catheter 112 to the maximum distance to which it can be advanced inside the patient's prostatic urethra from the apex of the patient's prostate gland towards the neck of the patient's urinary bladder. Thus, the advance limit switch 302 notifies the motion control microcontroller 328 not to advance the acoustic imaging catheter 112 any further. The retract limit switch 304 provides a notification to the motion control microcontroller 328 that the axial drive motor 306 has fully retracted the acoustic imaging catheter 112 to the maximum distance to which it can be retracted from the neck of the patient's urinary bladder towards the apex of the patient's prostate gland. Thus, the retract limit switch 304 notifies the motion control microcontroller 328 not to retract the acoustic imaging catheter 112 any further. The motion control microcontroller 328 can also initiate other machine functions through the I/O output pins. In an embodiment of the invention, the motion control microcontroller 328 is a PIC18F452 device with 32 KB ROM capacity, 1536B RAM capacity, 10MIPs 40 MHz Core and 256B EEPROM. It will be apparent to a person skilled in the art that the motion control microcontroller 328 is capable of performing many additional tasks, and that many other microcontrollers, microcomputers, ‘system-on-a-chip’ and other processing devices may be used in the invention.

The speech recognition microcontroller 330 includes a first speech recognition program module and a second speech recognition program module. The first speech recognition program module processes speech signals for controlling the motion of the rotational motor 310. The second speech recognition program module processes the speech signals for controlling motion of the axial drive motor 306. In an embodiment of the invention, the speech recognition microcontroller 330 is a dsPIC30F5013 device with 661(B ROM capacity, 4096B RAM capacity, 30MIPs 40 MHz Core and 1 KB EEPROM. It will be apparent to a person skilled in the art that the speech recognition microcontroller 330 is capable of performing many additional tasks, and that many other microcontrollers, microcomputers, ‘system-on-a-chip’ and other processing devices may be used in the invention.

In accordance with an embodiment of the invention, the motion control system 110 is powered by a power supply system. In this embodiment, the power supply system includes the power adapter 314 with safety approvals, such as UL, CUL, TUV, CE and CB. The power adapter 314 provides output voltage of 15V at maximum current rating of 3.3A. Maximum power output of the power adapter 314 is 50 Watts. The power adapter 314 provides power to the motion control system 110, the axial drive motor 306 and the rotational motor 310. The motion control system 110 itself has an internal power supply that converts some of the power to 5VDC to power the motion control microcontroller 328 and speech recognition microcontroller 330 and other electronic hardware while passing the higher voltage directly through to the axial drive motor driver 318 and the rotational motor driver 320 to power the axial drive motor 306 and the rotational motor 310. The power supply system is fused to prevent an overcurrent flow within the motion control system 110. In another embodiment, the voltages and currents may vary as needed or desired.

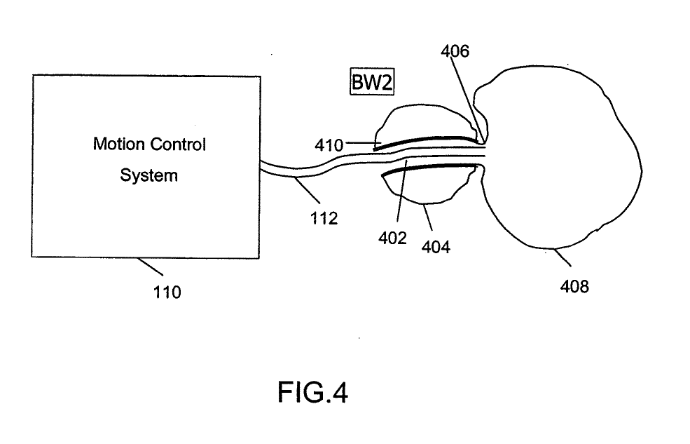

FIG. 4 illustrates a sectional anatomical view showing the acoustic imaging catheter 112 within a patient's prostatic urethra, in accordance with an embodiment of the invention. The acoustic imaging catheter 112 is connected to the motion control system 110. The acoustic imaging catheter 112 is moved inside the patient's prostatic urethra 402.

The axial motion of the acoustic imaging catheter 112 inside the patient's prostatic urethra 402 is controlled by the axial drive motor 306. In accordance with an embodiment of the invention, the acoustic imaging catheter 112 is moved inside the patient's prostatic urethra 402 such that the positional integrity of the acoustic imaging catheter 112 is maintained in relation to the neck 406 of a patient's urinary bladder 408. In an embodiment of the invention, the acoustic imaging catheter 112 can be provided with the linear displacement resolution of 0.000125 inch inside the patient's prostatic urethra 402. The acoustic imaging catheter 112 can be advanced towards the neck 406 of the patient's urinary bladder 408. The acoustic imaging catheter 112 can be retracted away from the neck 406 of the patient's urinary bladder 408 towards an apex 410 of the patient's prostate gland 404.

The acoustic imaging catheter 112 includes the ultrasonic transducer, which can be rotated at a selected rotational speed inside the acoustic imaging catheter 112. In an embodiment of the invention, the ultrasonic transducer is rotated at a speed of 100 rpm inside the acoustic imaging catheter 112. It will be apparent to a person skilled in the art that the ultrasonic transducer scans the patient's prostate gland 404 at predefined axial (longitudinal) intervals, each interval scan covering a transverse angular rotation of up to 360°. The ultrasonic transducer scans the anterior, posterior, inferior, and superior aspects of the patient's prostate gland 404. As the acoustic imaging catheter 112 is moved to different positions by advancement and retraction inside the patient's prostatic urethra 402, it produces multiple images (typically, 160 or more) of transverse (cross-sectional) specific slices of the patient's prostate gland 404, resulting in 360° imaging of the entire prostate gland 404 of the patient. The transverse slices can then be assembled into a three-dimensional image of the patient's prostate gland 404 for interactive viewing and analysis, by the imaging computer system 104.

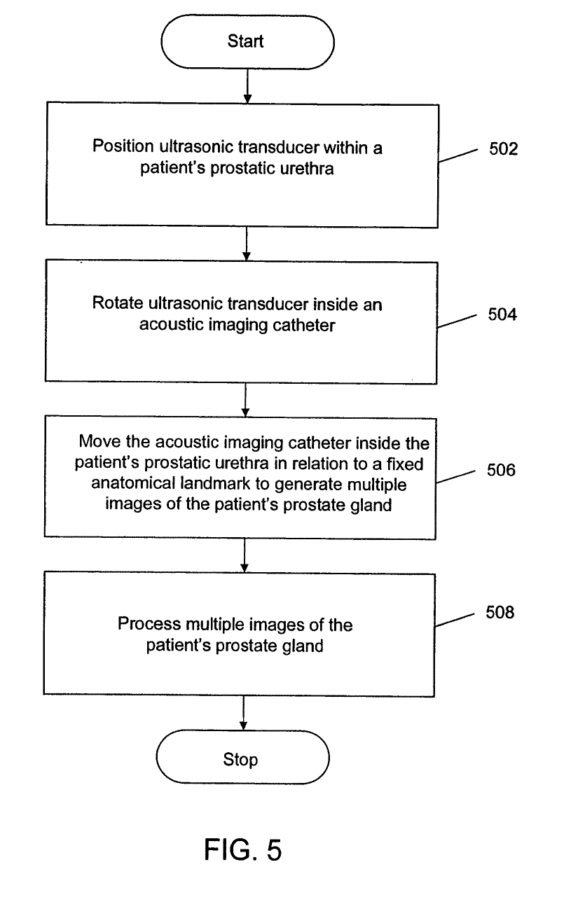

FIG. 5 is a flow chart illustrating a method for imaging a patient's prostate gland, in accordance with an embodiment of the invention. At step 502, the ultrasonic transducer is positioned within the patient's prostatic urethra. The ultrasonic transducer is positioned in such a manner within the patient's prostatic urethra that it enables the production of scan data representative of a section of the patient's prostate gland. At step 504, the ultrasonic transducer is rotated inside the acoustic imaging catheter 112. In an embodiment of the invention, the ultrasonic transducer is rotated at a rotational speed of 100 rpm. It will be apparent to those skilled in the art that other speeds may be selected, depending on the anatomical imaging requirements. At step 506, the acoustic imaging catheter 112 is moved to different positions inside the patient's prostatic urethra, in relation to the neck of the urinary bladder. This generates multiple transverse slice images of the selected sections of the patient's prostate gland.

In the various embodiments of the invention, the movement of the acoustic imaging catheter 112 inside the patient's prostatic urethra is automated. In accordance with one embodiment of the invention, the movement of the acoustic imaging catheter 112 inside the patient's prostatic urethra is controlled by the imaging computer system 104. In another embodiment of the invention, the movement of the acoustic imaging catheter 112 is manually controlled. At step 508, multiple transverse slice images of the patient's prostate gland are processed by the imaging computer system 104 to create a three-dimensional image of the patient's prostate gland. The three-dimensional image of the patient's prostate gland is displayed on the associated image viewing device 116.

It will be evident to a person ordinarily skilled in the art that one or more of the embodiments mentioned above provide the following advantages for ultrasound imaging of the patient's prostate gland. The embodiments of the invention enable two and three-dimensional imaging of the patient's prostate gland. The embodiments of the invention provide multiple arrays of transverse slice images of the selected section of the patient's prostate gland, which results in the complete scanning of the patient's prostate gland. The embodiments of the invention enable the storage of the patient's image data in the computer system, which can enable the retrieval of the required information anytime. The embodiments of the invention enable the rotation of the ultrasonic transducer inside the acoustic imaging catheter, which results in the scanning of the patient's prostate gland at predefined spatial and angular orientations. The embodiments of the invention can facilitate digitally positioned targeted biopsies based on image-apparent focal tissue abnormalities, potentially reducing the required number of tissue biopsy samples.

While the invention is susceptible to various modifications and alternative forms, specific examples thereof have been shown by way of example in the drawings and are herein described in detail. It should be understood, however, that the invention is not to be limited to the particular foams or systems or methods disclosed, but to the contrary, the invention is to cover all modifications, equivalents, and alternatives falling within the spirit and scope of the appended claims.

What is claimed is:

1. An ultrasound scanning system for imaging a patient's genitourinary system, the ultrasound scanning system comprising:
an acoustic imaging catheter capable of insertion within the patient's urethra;
a motion control system for controlling axial and rotational motion of the acoustic imaging catheter; and
an imaging computer system in communication with the acoustic imaging catheter and the motion control system, the imaging computer system outputting signals to the acoustic imaging catheter and processing signals received from the acoustic imaging catheter and generating image data for display on an associated imaging device,
wherein the image viewing device is positioned in a location allowing substantially simultaneous view of both a patient's pelvic region and the image viewing device.

2. The ultrasound scanning system according to claim 1 wherein the motion control system further comprises:
an interface for connecting the motion control system to the computer system;
a motion control micro controller for controlling the speed of a rotational motor and for controlling action of an axial drive motor;
a rotational motor encoder, the rotational motor encoder providing rotational speed information of the rotational motor and orienting a superior aspect of the image of the patient's prostate gland at the top of a display screen; and
a speech recognition micro controller for processing speech signals for controlling motion of the rotational motor and for processing speech signals for controlling motion of the axial drive motor and for general control of the imaging computer system.

3. The ultrasound scanning system according to claim 1 wherein the imaging computer system comprises means for entering patient specific data.

4. The ultrasound scanning system according to claim 1 wherein the imaging computer system comprises means for storing sonographic images of the patient's prostate gland on non-volatile media that can be included in a patient file.

5. The ultrasound scanning system according to claim 1 wherein the imaging computer system comprises a remote keypad for controlling the imaging of the patient's prostate gland.

6. The ultrasound scanning system according to claim 5 wherein the remote keypad comprises:
a spill-proof membrane keypad comprising keys for entering commands for imaging the patient's prostate gland; and
a display screen for displaying a plurality of data points in relation to imaging of the patient's prostate gland.

7. The ultrasound scanning system according to claim 6 wherein the display screen displays a plurality of programmable system control options in relation to imaging of the patient's prostate gland.

8. The ultrasound scanning system according to claim 1 wherein the imaging computer system comprises a program for controlling the motion control system.

9. An ultrasound scanning system for imaging a patient's prostate gland, the ultrasound scanning system comprising:
an acoustic imaging catheter wherein the acoustic imaging catheter comprises an ultrasonic transducer for scanning the patient's prostate gland;
an imaging computer system for processing signals from the ultrasonic transducer and creating multiple transverse slice images which can be assembled into a three dimensional rendering of the patient's prostate gland wherein the imaging computer system comprises:
means for storing the multiple transverse slice images of the patient's prostate gland in relation to the position of the acoustic imaging catheter relative to the neck of the patient's urinary bladder;
means for displaying image data relating to the patient's prostate gland;
means for controlling axial and rotational motion of the acoustic imaging catheter; and
a remote keypad for controlling the imaging of the patient's prostate gland and limiting interaction between a physician and the ultrasound scanning system to minimize potential for errant instructions; and
a motion control system comprising:
an interface for connecting the motion control system to the imaging computer system;
a motor for moving the acoustic imaging catheter inside the patient's prostatic urethra wherein the acoustic imaging catheter is moved to different positions inside the patient's prostatic urethra in relation to the neck of the patient's urinary bladder;
a motion platform for rotating the ultrasonic transducer inside the acoustic imaging catheter;
a motion control micro controller for controlling the rotational speed of the motor and the axial motion of the acoustic imaging catheter;
a rotational motor encoder for providing rotational speed information of the motor and orienting a superior aspect of the image of the patient's prostate gland at the top of a display screen; and
a speech recognition micro controller for processing speech signals for controlling motion of the motor and control of the imaging computer system.
